(19) United States
(12) Patent Application Publication
(10) Pub. No.: US 2021/0330489 A1
(43) Pub. Date: Oct. 28, 2021

(54) GROIN AREA WARMING DEVICE

(71) Applicant: Douglas Clemons, Vernon, AZ (US)

(72) Inventor: Douglas Clemons, Vernon, AZ (US)

(21) Appl. No.: 16/861,113

(22) Filed: Apr. 28, 2020

(51) Int. Cl. A61F 7/02; A61F 7/00

(52) U.S. Cl. CPC A61F 7/02; A61F 2007/0236; A61F 2007/0048; A61F 7/007

(57) ABSTRACT

A groin area warming device comprises a sleeve having a first end and a second end, a first confined area between the first end and the second end, and a second confined area at the second end of the sleeve. The first confined area comprises a heat pad and the second confined area comprises a power source. The second confined area folds over a portion of the first confined area on fastening the device to the apparel of the user. The power source is operatively coupled to the head pad to provide power to the head pad. A control switch is operatively coupled to the head pad and the power source to control operation of the device. The control switch is further configured to control the generation of heat at the heat pad or application of heat to at least a portion of the groin area of the user.

### TECHNICAL FIELD

The present disclosure relates generally to a warmer device, and more particularly, to a localized warmer device for warming at least a portion of the groin area of a user.

### BACKGROUND

People involved in sporting activities, for example motorcycling, are often subject to severe weather. Even those who wear leather riding pants or other protective riding clothing may find that the button or zipper closures allow wind and wet weather to penetrate sensitive areas. Some motorcyclists often wear chaps for protection from the cold weather. However, chaps leave the groin and inner thigh areas completely unprotected. These sensitive areas become cold easily, especially when wet, causing great discomfort. This is especially true under hazardous driving conditions. Discomfort is known to cause stress, fatigue and inattention while operating a motor vehicle, leading to unfortunate accidents.

In addition to people involved in sporting activities, situations where people working in very hostile environments are not able to remove or add to their protective clothing and/or suits if they become too cold, such as firemen, drivers, and the like. Conventional solutions involve utilizing articles of garment to keep the wearer warm. However, shortcomings include lack of adherence of garment to the body of the wearer, undesirable aerodynamic effects, and obstruction to wearer movements.

### SUMMARY

A groin area warming device comprises a sleeve having a first end and a second end, a first confined area between the first end and the second end, and a second confined area at the second end of the sleeve. The first confined area comprises a heat pad and the second confined area comprises a power source. The second confined area folds over a portion of the first confined area on fastening the device to the apparel of the user. The power source is operatively coupled to the head pad to provide power to the head pad. A control switch is operatively coupled to the head pad and the power source to control operation of the device. The control switch is further configured to control the generation of heat at the heat pad or application of heat to at least a portion of the groin area of the user.

### BRIEF DESCRIPTION OF THE DRAWINGS

FIG. 1 exemplarily illustrates a perspective view of a groin area warming device, according to an embodiment of the present invention.

FIG. 2 exemplarily illustrates the groin area warming device positioned at a groin region of a user, according to an embodiment of the present invention.

FIG. 3 exemplarily illustrates a see-through view of the groin area warming device, according to an embodiment of the present invention.

FIG. 4 exemplarily illustrates a see-through view of the groin area warming device, according to another embodiment of the present invention.

FIG. 5 exemplarily illustrates the components of the groin area warming device, according to an embodiment of the present invention.

FIG. 6 exemplarily illustrates the groin area warming device being fastened to apparel of the user, according to an embodiment of the present invention.

FIG. 7 exemplarily illustrates the groin area warming device worn by the user, according to an embodiment of the present invention.

FIG. 8 exemplarily illustrates a rear view of the groin area warming device, according to an embodiment of the present invention.

### DETAILED DESCRIPTION OF EXAMPLE EMBODIMENTS

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

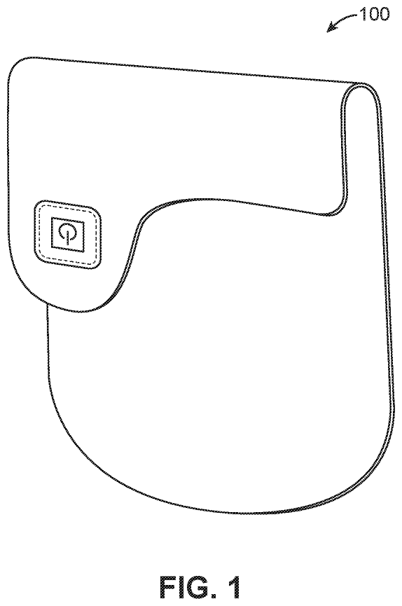

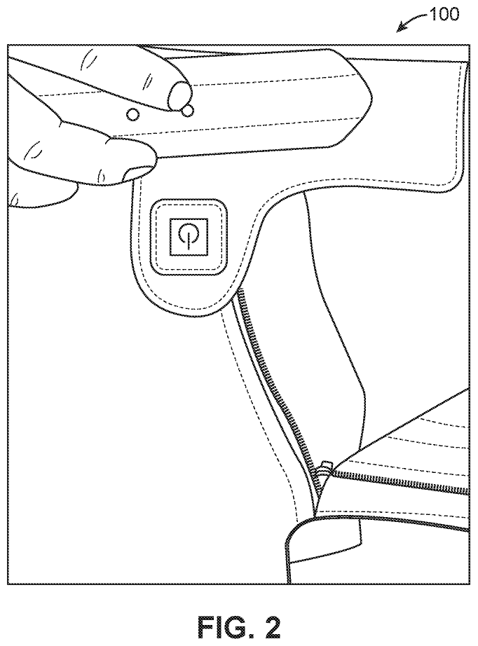

FIG. 1 exemplarily illustrates a perspective view of a groin area warming device 100. The configuration of the device 100 allows a user to position or fasten the device 100 at the groin region of the user. The device 100 is slipped underneath apparel of the user and positioned to be in contact with the user's groin region, as shown in FIG. 2. The device 100 is configured to warm at least a portion of the groin area of the user.

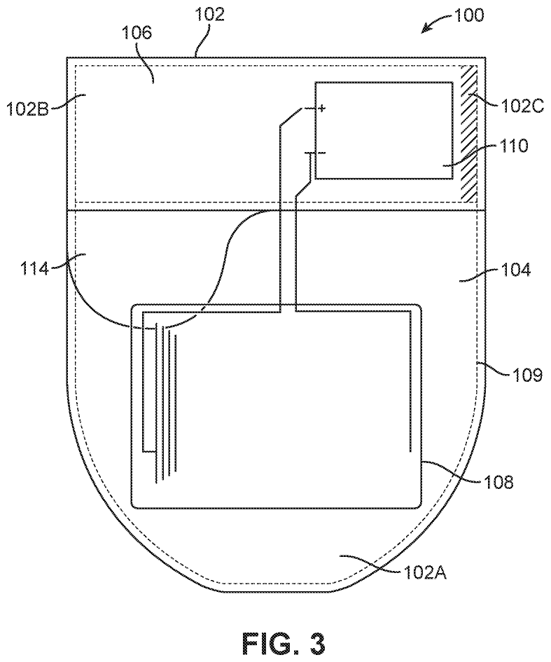

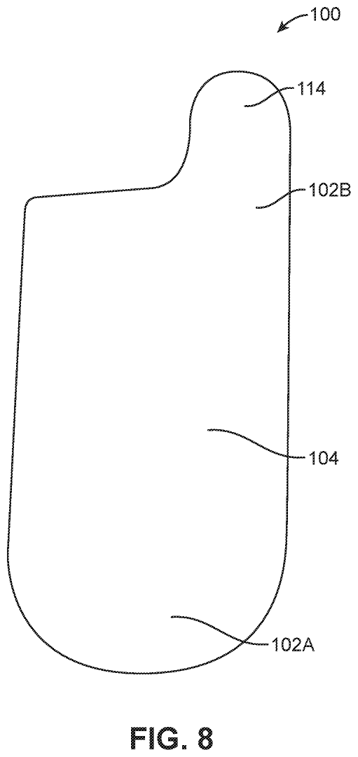

FIG. 3 exemplarily illustrates a see-through view of the groin area warming device 100. The device 100 comprises a sleeve 102 having a first end 102A and a second end 102B, a first confined area 104 between the first end 102A and the second end 102B, and a second confined area 106 at the second end 102B of the sleeve 102 (shown in FIG. 3 and FIG. 8). The first confined area 104 comprises a heat pad 108 and the second confined area 106 comprises a power source 110.

The second confined area 106 folds over at least a portion of the first confined area 102 on positioning the device 100 to the apparel of the user. The power source 110 is operatively coupled to the head pad 108 to provide power to the head pad 108. In one embodiment, the first confined area 104 is incorporated with any number of head pads 108. Preferably, the device 100 is configured to generate heat in the temperature range of about 80-degree Fahrenheit to 120-degree Fahrenheit. In various exemplary embodiment, the temperature to which the device 100 is heated may be controlled, thus allowing the user to select a temperature level most comfortable to the user at the time of the usage. In still other embodiments, the temperature can be controlled using a built-in thermostat. In an embodiment, the heat pad 108 may be a 4″ by 4″ heat pad 108 powered by a 5V 3A power source 110.

In an embodiment, the sleeve 102 may be made of a genuine or synthetic leather material. However, the sleeve 102 is not limited to be made of this material and may be made of any material suitable for warming purposes. The sleeve 102 is a pouch-type sleeve that is configured to house the heat pad 108 as well as the power source 110. In one embodiment, the second end 102B of the sleeve 102 is a flap type portion incorporated with a hook and loop fastener 102C provided at one of the lateral ends of the second confined area 106 for providing access to the interior of the second confined area 106. The reason the hook and loop fastener 102C are provided to the second confined area 106 to facilitate the removal and insertion a charging cable to recharge the power source 110 as and when the power source 110 is exhausted. More specifically, the sleeve 102 is provided with number of stitches 109 for generating a compartmentalized configuration within the sleeve 102 to hold the different components of the device 100 and also to provide an aesthetic appearance. In one embodiment, the heat pad 106 may be a washable type heat pad.

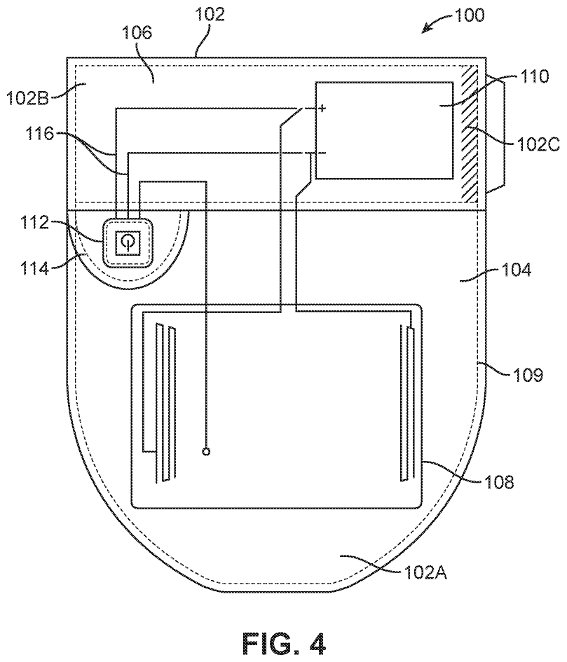

FIG. 4 exemplarily illustrates a see-through view of the groin area warming device 100, according to another embodiment of the present invention. The device 100 comprises a control switch 112. The control switch 112 is operatively coupled to the head pad 106 and the power source 110 to control the operation of the device 100. The control switch 112 is disposed over a tab portion 114 that extends from the second end 102B of the sleeve 102. The tab portion 114 allows the wearer to manipulate the flap or the second end 102B of the sleeve 102, thereby allowing the user to position the device 100 near the groin region in a position that is the most comfortable to the user. In an embodiment, the control switch 112 is configured to turn on and turn off the device 100. In this embodiment, the control switch 112 can be a push-button switch, for example, or another type of on/off switch.

In some embodiments, the control switch 112 can be further configured to control the level of heat at the heat pad 108 application of heat to at least a portion of the groin area of the user. Using the control switch 112, the user may adjust the temperature to which the device 100 is heated, thereby providing maximum comfort to the user. As an example, in these embodiments, the control switch 112 could be a multi-level switch or a dimmer switch to control the voltage level of the device 100, preferably, allowing control of temperature within a range of about 80 degrees Fahrenheit to 120 degrees Fahrenheit. The control switch 112 and the power source 110 located at the second end 102B are folded over the user's apparel, which prevents contact of batteries with the user's skin.

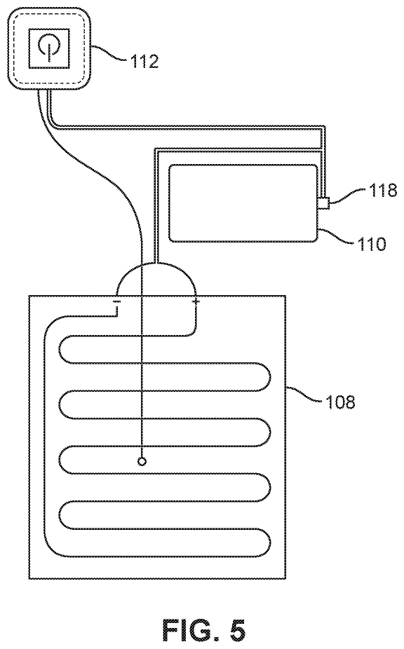

FIG. 5 exemplarily illustrates the components of the groin area warming device 100, according to an embodiment of the present invention. The control switch 112, the power source 110, and heat pad 108 are operatively connected to one another via a plurality of wires 116. As shown, the control switch 112 is a push-button on/off switch. However, the device 100 could include a thermostat (not shown) integral to the heat pad 108 to measure and regulate the temperature of the heat pad 108 by connecting and disconnecting the electricity/power supplied to the heat pad 108. Alternatively, the control switch 112 could include a multi-level or a dimmer switch to regulate the voltage. In one embodiment, the power source 110 includes one or more rechargeable batteries. The device 100 includes one or more ports 118 to charge the power source 110. In one embodiment, the port 118 may be a USB port or any other type of port that facilitate the charging of rechargeable batteries. The port 118 facilitates the charging of the device 100 via electrical connection to an AC outlet or a power bank using the conventionally know charging cables, e.g., USB cables.

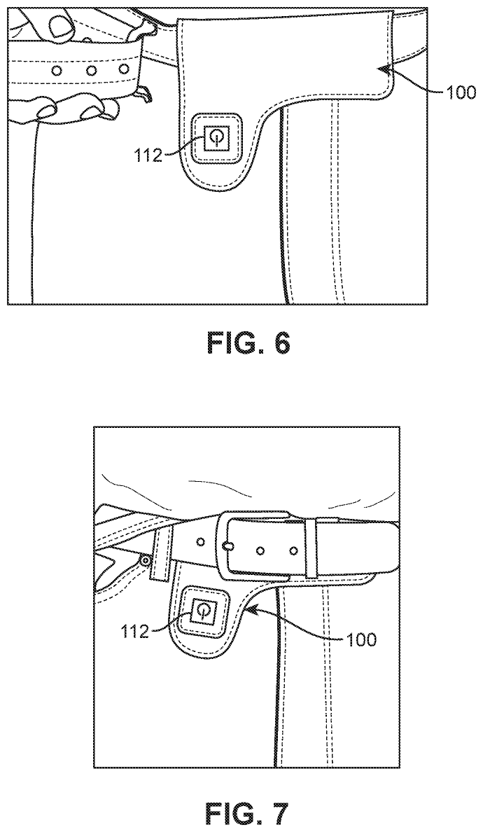

FIG. 6 exemplarily illustrates the groin area warming device 100 being positioned within the apparel of the user. The device 100 is slipped underneath the apparel of the user and positioned to be in contact with the user's groin region. The second end 102B of the sleeve 102 is folded over the apparel. More specifically, the first confined area 104 includes which the heat pad 108 is placed adjacent the groin area of the user, while the second confined area 106 is folded over the apparel of the user. The belt of the user is used to fasten the device 100 and maintain the position of the device 100, as shown in FIG. 7.

FIG. 8 exemplarily illustrates a rear view of the groin area warming device 100 having the sleeve 102 made of leather. The outer leather surface facilitates the cleaning of the device 100 easily without causing damage to the components of the device 100. The device 100 also provides comfortable feel and touch to the user, as the leather is pliable and soft. The leather sleeve 102 is adapted to block wind to the user's groin area. The device 100 is suitable for people working in very hostile environments, who are not able to remove or add to their protective clothing and/or suits if they become too cold. Particularly, people involved in sports activities such as motorcyclists.

Although the features, functions, components, and parts have been described herein in accordance with the teachings of the present disclosure, the scope of coverage of this patent is not limited thereto. On the contrary, this patent covers all embodiments of the teachings of the disclosure that fairly fall within the scope of permissible equivalents.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A warmer device, comprising:
a sleeve having a first end and a second end to position on at least a portion of a groin area of a user;
a first confined area between the first and second end including at least one heat pad;
a second confined area at the first end including a power source, wherein the power source is configured to provide power to the heat pad, and
a control switch operatively coupled to the heat pad and power source, wherein the control switch is configured to control operation of the device.

2. The warmer device of claim 1, wherein the second end of the sleeve comprises at least one fastener member to secure the device onto the user.

3. The warmer device of claim 1, wherein the second confined area is adapted to fold over and rest on at least a portion of the first confined area.

4. The warmer device of claim 1, wherein the control switch is disposed at the second end of the sleeve.

5. The warmer device of claim 1, wherein the control switch is configured to turn on and turn off the device.

6. The warmer device of claim 1, wherein the control switch is configured to control the heat applied to at least a portion of the groin area of a user.

7. The warmer device of claim 1, wherein the first confined area rests over the portion of the groin area of the user and the second confined area folds over an apparel being worn by the user to maintain a position of the first confined area.

8. The warmer device of claim 1, wherein the sleeve is made of leather.

9. The warmer device of claim 1, wherein the sleeve is a pouch-type sleeve.

10. The warmer device of claim 1, wherein the power source comprises one or more rechargeable batteries.

11. The warmer device of claim 1, comprises one or more ports for charging the rechargeable batteries.

12. The warmer device of claim 1, wherein the distal end of the sleeve is a flap.

13. The warmer device of claim 1, comprises a tab extending from the distal end.

14. The warmer device of claim 1, wherein the control switch is disposed on the tab.
